(19) United States
(12) Patent Application Publication
(10) Pub. No.: US 2017/0119974 A1
(43) Pub. Date: May 4, 2017

(54) NEEDLES AND RELATED ASSEMBLIES AND METHODS

(71) Applicant: Custom Medical Applications, Inc., Johnstown, NY (US)

(72) Inventor: N. Sandor Racz, Farmers Branch, TX (US)

(21) Appl. No.: 15/249,092

(22) Filed: Aug. 26, 2016

Related U.S. Application Data
(60) Provisional application No. 62/250,866, filed on Nov. 4, 2015.

(51) Int. Cl. A61M 5/32

(52) U.S. Cl. CPC A61M 5/32; A61M 5/3286; A61M 2207/10

(57) ABSTRACT

Needles include a cannula defining a bore within the cannula, a distal opening of the bore of the cannula, and one or more of at least one protrusion or an enlarged rounded surface on a proximal surface of the needle proximate the distal opening and at least one chamfered surface positioned proximate a tip of the cannula. Methods include forming at least one protrusion on a distal portion of a needle bordering a distal opening of the needle.

### CROSS-REFERENCE TO RELATED APPLICATION

This application claims the benefit of U.S. Provisional Patent Application Ser. No. 62/250,866, filed Nov. 4, 2015, the disclosure of which is hereby incorporated herein in its entirety by this reference.

### TECHNICAL FIELD

The disclosure relates generally to the field of medical devices and related methods. In particular, the disclosure relates to needles (e.g., introducer needles) that may be utilized to administer anesthetics and analgesic medications and/or utilized to deploy another device, such as, for example, a catheter, a lead, or other device, proximate portions of the nervous system of a subject and related assemblies and methods.

### BACKGROUND

Implantable medical devices (e.g., medical therapy delivery devices), such as catheters and leads, may be employed for a variety of therapeutic and diagnostic purposes. Controlled placement and retention of such therapy delivery elements in a subject is highly desirable as precise placement and retention should result in improved therapeutic efficacy and/or reduced side effects. However, as introducer needles generally have a sharp point or chisel-shaped sharp point and are designed to cut tissue for insertion into a subject, the needle may also act to damage the catheter or lead as it threaded through the lumen of the introducer needle and deployed within the subject. For example, sharp edges and surfaces of an introducer needle surrounding a distal opening of the introducer needle may damage (e.g., shear, cut, and/or skive) a catheter or leads as it exits the lumen of the needle during deployment within a subject. Such damage may reduce or compromise the functionality of the catheter or leads causing partially or total failure of the device. Further, catheters or leads inserted through the needle may be cut or severed by the sharp edges of the sharp points or chisel-shaped sharp points on the end of the needles making it difficult to remove the catheter from the needle or causing the partially severed catheter to traumatize surrounding tissue.

Moreover, many conventional introducer needles have a relatively wide cutting edge due to the large opening of the orifice or bore formed in the introducer needle needed to introduce catheters and leads. However, such a relatively wide cutting edge is often traumatic to tissues of a subject when the introducer needle is inserted into the subject. In particular, such a relatively wide cutting edge may act like a scalpel edge with a wide cutting surface and may cut and/or damage the subject's tissue during insertion.

### BRIEF SUMMARY

Described are introducer needles, introducer needle assemblies, methods of introducing a medical device within a subject, and other related assemblies, devices, and methods. Such introducer needles may be utilized to administer one or more therapies to a subject (e.g., anesthetics, analgesic medications) and/or to introduce and/or position one or more associated devices (e.g., a catheter and/or lead) within the subject while at least a portion of the introducer needle is positioned within (e.g., resident in) the subject.

Disclosed is an introducer needle. The introducer needle includes a cannula defining a bore within the cannula, the bore configured to pass at least one associated medical device through the bore and into a subject, a beveled end defining a distal opening of the bore of the cannula, and at least one protrusion on a proximal surface of the beveled end proximate the distal opening.

Also disclosed is a needle comprising a cannula defining a bore within the cannula, a beveled end defining a distal opening of the bore of the cannula and having a sharp tip, and at least one protrusion on a proximal surface of the beveled end proximate the distal opening.

Further disclosed is a needle comprising a cannula defining a bore within the cannula, a beveled end defining a distal opening of the bore of the cannula and having a sharp tip, and at least one chamfered surface extending between a surface defining the beveled end and an outer circumferential surface of the cannula and positioned proximate the sharp tip. In some embodiments, the at least one chamfered surface comprises two chamfer surfaces positioned on either side of the sharp tip of the beveled end of the introducer needle. In some embodiments, the needle further includes at least one protrusion on a proximal surface of the beveled end proximate the distal opening.

Further disclosed is a needle comprising a cannula defining a bore within the cannula. The bore is configured to pass at least one associated medical device through the bore and into a subject. The needle further includes a distal portion defining a distal opening of the bore of the cannula and at least one enlarged rounded surface on the distal portion proximate the distal opening. In some embodiments, the distal opening is positioned in a plane that is positioned at an oblique angle relative to a longitudinal axis of the cannula. In some embodiments, the at least one enlarged rounded surface comprises at least one rounded protrusion positioned on a proximal portion of the distal portion of the needle defining a rearward portion of the distal opening.

Also disclosed are medical device assemblies including a medical device configured to have at least a portion thereof positioned within subcutaneous tissue of a subject and a needle, according to an embodiment of the instant disclosure, for inserting at least a portion of the medical device within the subject.

Also disclosed are methods of forming and utilizing introducer needles and introducer needle assemblies according to the disclosure.

For example, a method of forming a needle may include providing a cannula having a bore within the cannula, configuring the bore to pass at least one associated medical device through the bore and into a subject, defining a distal opening of the bore of the cannula at a distal end of the needle, and forming at least one protrusion on a surface of a distal end of the cannula bordering the distal opening of the cannula.

### BRIEF DESCRIPTION OF THE FIGURES

FIG. 1 depicts an introducer needle in accordance with an embodiment of the disclosure.

FIG. 2 depicts an enlarged view of a distal portion of an introducer needle in accordance with an embodiment of the disclosure.

FIG. 3 depicts an enlarged view of a distal portion of an introducer needle in accordance with an embodiment of the disclosure.

FIG. 4 depicts an enlarged view of a distal portion of an introducer needle in accordance with an embodiment of the disclosure.

FIG. 5 depicts an enlarged view of a distal portion of an introducer needle in accordance with an embodiment of the disclosure.

FIG. 6 depicts an enlarged view of a distal portion of an introducer needle in accordance with an embodiment of the disclosure.

FIG. 7 depicts a cross-sectional view of the distal portion of the introducer needle of FIG. 6.

FIG. 8 depicts an enlarged cross-sectional view of a distal portion of an introducer needle.

FIG. 9 depicts an enlarged cross-sectional enlarged view of a portion of the distal portion of the introducer needle of FIG. 6 superimposed over an enlarged cross-sectional enlarged view of a portion of the distal portion of the introducer needle of FIG. 8.

FIG. 10 depicts a medical device assembly including a medical device and a needle (e.g., a medical device being deployed from a needle) in accordance with and embodiment of the disclosure.

### DETAILED DESCRIPTION

Illustrations presented herein are not necessarily meant to be actual views of any particular device, assembly, system, method, or components thereof, but are merely idealized representations, which are employed to describe embodiments of the disclosure. Additionally, elements common between figures may retain the same numerical designation.

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

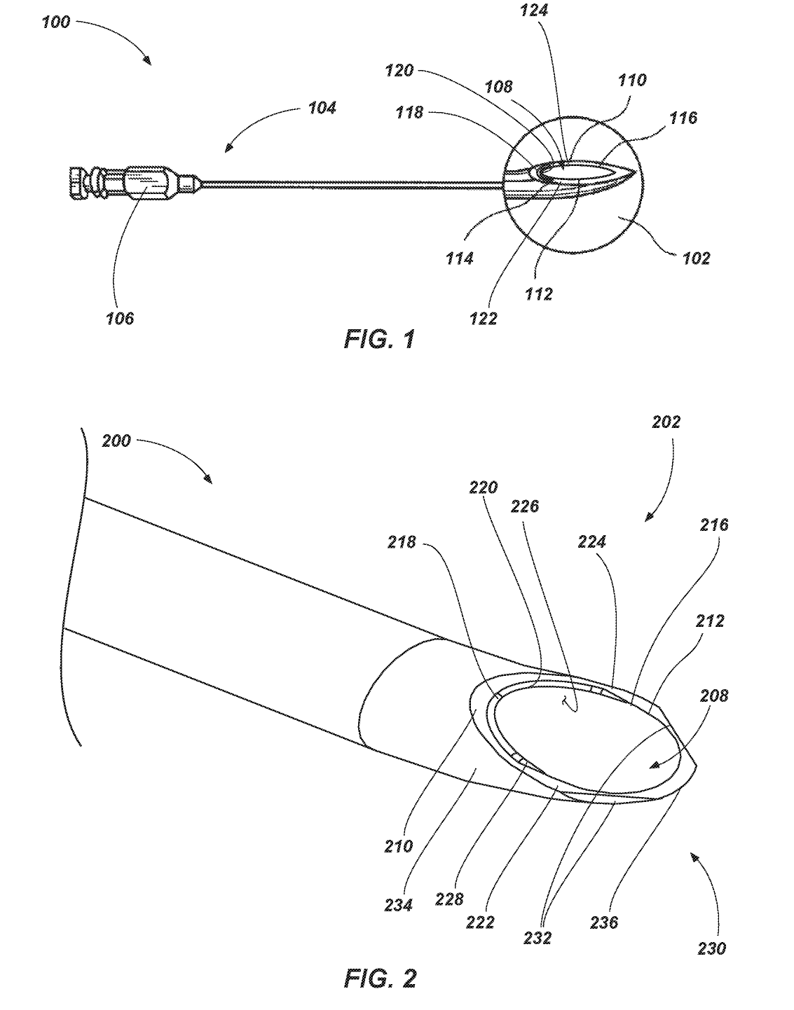

FIG. 1 depicts an introducer needle 100 (e.g., a Tuohy needle). As shown in FIG. 1, the introducer needle 100 is shown with a point or distal portion 102 of the introducer needle 100 shown in an enlarged view for clarity. The introducer needle 100 comprises a cannula defining a bore 108 in the cannula. The cannula may have a longitudinal axis (e.g., centerline) extending along the length of the cannula (e.g., along and within the bore 108).

As depicted, the introducer needle 100 may have a Huber point with a medium bevel. In some embodiments, the Huber point is utilized to assist in direction of the medical device and/or treatment deployed through the introducer needle 100 in the desired direction and to help prevent impingement on various internal structures (e.g., subarachnoid structures) and to facilitate the passage of the medical devices and/or treatments in the desired direction.

While FIG. 1 depicts a Tuohy needle, in other embodiments, the introducer needle may be formed as any suitable needle, such as, for example, a modified Tuohy needle (e.g., a Tuohy-Flowers needle) and other needles for use with a portion of a subject's nervous system (e.g., the peripheral nervous system and/or the central nervous system). For example, the introducer needle may comprise an epidural needle, such as, for example, a Crawford needle, a Hustead needle, a Weiss needle, a Sprotte needle, a Barker needle, etc.

In some embodiments, the introducer needle 100 may be utilized to administer one or more therapies to a subject (e.g., by directly delivering anesthetics and/or analgesic medications to a selected location within the subject). In some embodiments, the introducer needle 100 may be utilized to introduce and/or position one or more associated devices (e.g., a catheter, lead, and/or lead extension) within the subject while at least a portion of the introducer needle is positioned within (e.g., resident in) the subject. For example, the introducer needle 100 may be utilized to introduce and/or position a medical device comprising, for example, a diagnostic device, a monitoring device, a therapeutic device, or combinations thereof. In some embodiments, such a medical device may comprise a medical therapy delivery device, a medical device configured to sense a parameter of the subject, a medical device configured to diagnose a condition, a medical device configured to sample one or more tissues and/or fluids from a subject, or combinations thereof. In some embodiments, at least a portion of the medical device (e.g., a catheter, a lead, or lead extension) is positioned proximate the nervous system of a subject (e.g., proximate the spinal cord or canal, brain, and/or peripheral nervous system).

A proximal end 104 of the introducer needle 100 may be connected to a needle hub 106. The needle hub 106 is typically configured to remain outside a subject during a procedure. The needle hub 106 may be configured for handling by a practitioner, such as, for example, by including a portion curved to accommodate a grip, by including ribs or other gripping members to facilitate manipulation of the introducer needle 100. The needle hub 106 may also be configured for connection to another structure or device, such as, for example, by including a LUER-LOK® connection, a Luer-Slip connection, or a threaded connection. The needle hub 106 may be configured to enable other structures, devices, or substances to pass through the needle hub 106 into the bore 108 of the introducer needle 100.

The distal portion 102 may include a bevel 110 formed or shaped to define an opening 112 at a distal end of the bore 108 of the introducer needle 100 having a distal portion 114 and a proximal portion 116. The distal portion 102 of the introducer needle 100 is curved upward to allow fluid being ejected therefrom or any medical devices threaded therethrough to exit in an expected direction. Typically, a slot or other indicator in, for example, the needle hub 106 indicates the direction of the bevel 110.

As depicted, the introducer needle 100 includes one or more features (e.g., an enlarged surface, an enlarged rounded surface, an enlarged blunted surface, a protrusion 118) formed proximate the opening 112 (e.g., distal opening) of the bore 108 at the distal portion 102 of the introducer needle 100. For example, the protrusion 118 may be positioned at a trailing or a proximal surface or portion 116 of the bevel 110 of the introducer needle 100 proximate or extending about the opening 112. Stated in another way, the protrusion 118 may be positioned at a trailing or proximal surface or end 120 of the portion of the introducer needle 100 defining the opening 112 (e.g., the bevel 110). The protrusion 118 may extend outward and/or upward (e.g., at least partially radially outward) from an outer surface of introducer needle 100 (e.g., an outer surface of the bevel 110).

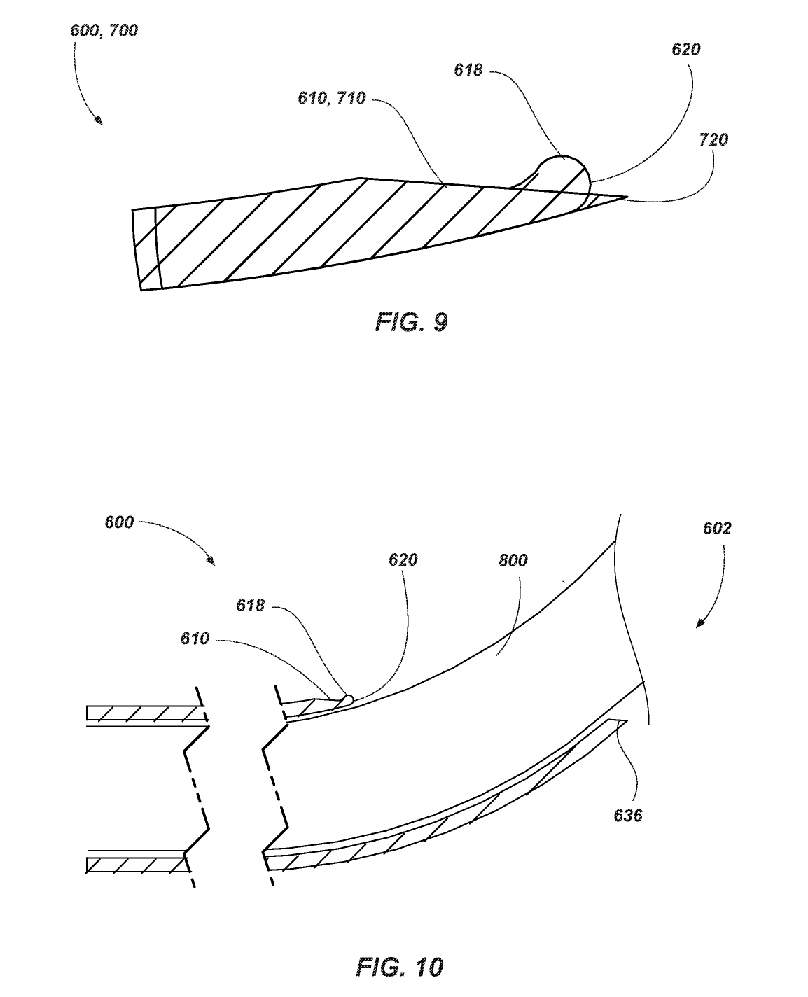

Generally, the sharp inside edge of the bevel 110, particularly, the trailing or proximal end 120 of the portion of the introducer needle 100 defining the opening 112, increases the hazard of shearing, cutting, and skiving of portions of a medical device (e.g., polymer tubing) utilized with introducer needle 100 (see, e.g., a sharp trailing or proximal end 720 of an introducer needle 700 depicted in FIGS. 8 and 9). This hazard may be increased if it is attempted to withdraw the medical device without first removing the introducer needle 100. While, in some embodiments, portions of the sharp inside edge of the bevel are blunted during manufacture (e.g., by filing down, electro-polishing, bead blasting, grinding with micro-conical grinding points, and/or reaming the sharp inside edge of the bevel), the problem remains.

As opposed to the above-listed conventional methods of removing material from the introducer needle 100 (e.g., from inner surfaces of the introducer needle 100) proximate the opening 112 to reduce the probability of damaging an associated medical device with the sharp inside edge of the bevel 110, embodiments of the disclosure may employ the addition of material to the introducer needle 100 proximate the opening 112 (e.g., to define an enlarged surface and/or a protrusion) to reduce the probability of damaging an associated medical device with the sharp inside edge of the bevel 110. For example, protrusion 118, which may be formed to surround at least a portion of the opening 112 (e.g., surrounding a quarter of the opening 112, surrounding a third of the opening 112, surrounding half of the opening 112, surrounding a three-fourths of the opening 112, surrounding an entirety of the opening 112), may act to provide a relatively more smooth surface (e.g., as compared to the sharp inside edge of a bevel lacking such a feature) for the medical device to contact and/or move or translate along (e.g., slide) during a procedure (e.g., during deployment and/or retraction from the introducer needle 100).

The enlarged or rounded heel of the bevel 110 of the introducer needle 100 enables for pressure applied to the surface of the bevel 110 with the medical device to be more evenly distributed across contacting portions of the medical device (e.g., a polymer tube) and a portion of the introducer needle 100 (e.g., the bevel 110). Such an effect enables the medical device to slide along the protrusion 118 during placement, adjustment, and/or removal of the medical device without significantly damaging the medical device.

In embodiments where the protrusion 118 is positioned primarily on the inner, non-cutting surface of the needle tip (e.g., on the heel or proximal end of the bevel 110), the protrusion 118 will have minimal to no effect on the penetration force of the introducer needle 100 design and can be implemented on any introducer needle format (e.g., formed during manufacture or retrofitted on existing needles).

As discussed above, in conventional introducer needles, the rear heel or proximal end of the bevel bounding the opening is rounded off to reduce the damage caused to an associated medical device deployed through the introducer needle. In order to allow for the rounding off of this rear heel or proximal end of the bevel, a cannula with a thicker wall is required so that there is enough material to round down without creating even sharper edges. Accordingly, relatively thinner walled cannulas are not implemented in introducer needle designs. However, embodiments of the instant disclosure enable the use of thinner walled cannulas as no rounding off of the rear heel or proximal end of the bevel is required in such needles. Such thinner walled cannulas enable the introducer needle to have a larger inner diameter without having to increase the overall gauge or outer diameter of the needle.

As further depicted in FIG. 1, the protrusion 118 may comprise a continuous structure extending from a first lateral side 122 of the opening 112, along the proximal end 120 of the opening 112, and to a second lateral side 124 of the opening 112 (e.g., opposing the first lateral side 122). For example, the continuous structure may define a continuous rounded surface having a generally wedge-shape (e.g., having a first relatively more narrow end and an opposing relatively more wider end), such as, a substantially C-shape, a substantially V-shape, a substantially rounded wedge-shape any other suitable type of wedge-shape.

In some embodiments, the protrusion 118 is formed by one or more of material applied to the introducer needle 100 (e.g., through a welding process or other deposition or thermal process) and material of the introducer needle 100. For example, a welding process or micro-welding process (e.g., electron-beam welding process, pulse arc welding processes, such as gas tungsten arc welding (TIG or GTAW welding) and micro gas tungsten arc welding (micro-GTAW) (also known as micro tungsten inert gas (micro-TIG) welding), gas metal arc welding, (MIG welding), flux-shielded arc welding, oxygen-acetylene torch welding, laser beam welding, such as pulsed laser welding or deposition, resistance discharge welding processes, capacitive discharge welding processes, etc.) may be utilized to build up the material on the introducer needle 100 to form the protrusion 118. In some embodiments, the protrusion 118 comprises material similar to that of the introducer needle 100 (e.g., medical grade stainless steel) and/or may comprise a dissimilar material (e.g., a different metal, polymer, etc.).

FIG. 2 depicts an enlarged view of a distal portion 202 of an introducer needle 200. The introducer needle 200 may be similar to and include one or more of the same features and functioning, as well as being formed by similar methods, as the introducer needle 100 discussed above with reference to FIG. 1. As shown in FIG. 2, the distal portion 202 of the introducer needle 200 may include a bevel 210 formed or shaped to comprise an opening 212 at a distal end of a bore 208 of the introducer needle 200. The distal portion 202 is curved upward to allow fluid being ejected therefrom or any medical devices threaded therethrough to exit in an expected direction.

As depicted, the introducer needle 200 includes one or more features (e.g., protrusion 218) formed proximate the opening 212 (e.g., at, bordering, forming a boundary of an opening 216) of the bore 208 at the distal portion 202 of the introducer needle 200. For example, the protrusion 218 may be positioned directly at a proximal surface or end 220 (e.g., the heel or trailing end of the bevel 210) of the portion of the introducer needle 200 defining the opening 212 (e.g., the bevel 210). In some embodiments, the protrusion 218 also at least partially defines the opening 212.

In some embodiments, the protrusion 218 extends into the opening 216, thereby defining at least a portion of the circumference of the opening 216. In some embodiments, the protrusion 218 extends into the opening 216 and partially onto an inner surface 226 of the introducer needle 200 defining the bore 208.

As above, the introducer needle 200 employs the addition of material to form the protrusion 218 of the introducer needle 200 proximate the opening 212 to reduce the probability of damaging an associated medical device with the sharp inside edge of the bevel 210. The protrusion 218, which, as depicted, may surround about half or more of the opening 212, acts to provide a relatively more smooth surface for a medical device to contact and/or move or translate along (e.g., slide) during a procedure (e.g., during deployment and/or retraction from the introducer needle 200) as compared to conventional introducer needles.

As further depicted in FIG. 2, the protrusion 218 may comprise a continuous structure (e.g., a continuous rounded surface having a substantially wedge-shape) extending at a junction between the bevel 210 (e.g., a surface defining the bevel 210) and the inner surface 226 of the introducer needle 200 defining the bore 208 from a first lateral side 222 of the opening 212, along the proximal end 220 of the opening 212, and to a second lateral side 224 of the opening 212 (e.g., opposing the first lateral side 222). In some embodiments, ends 228 of the protrusion 218 may be tapered smoothly into a surface of the introducer needle 200 (e.g., the surface defining the bevel 210).

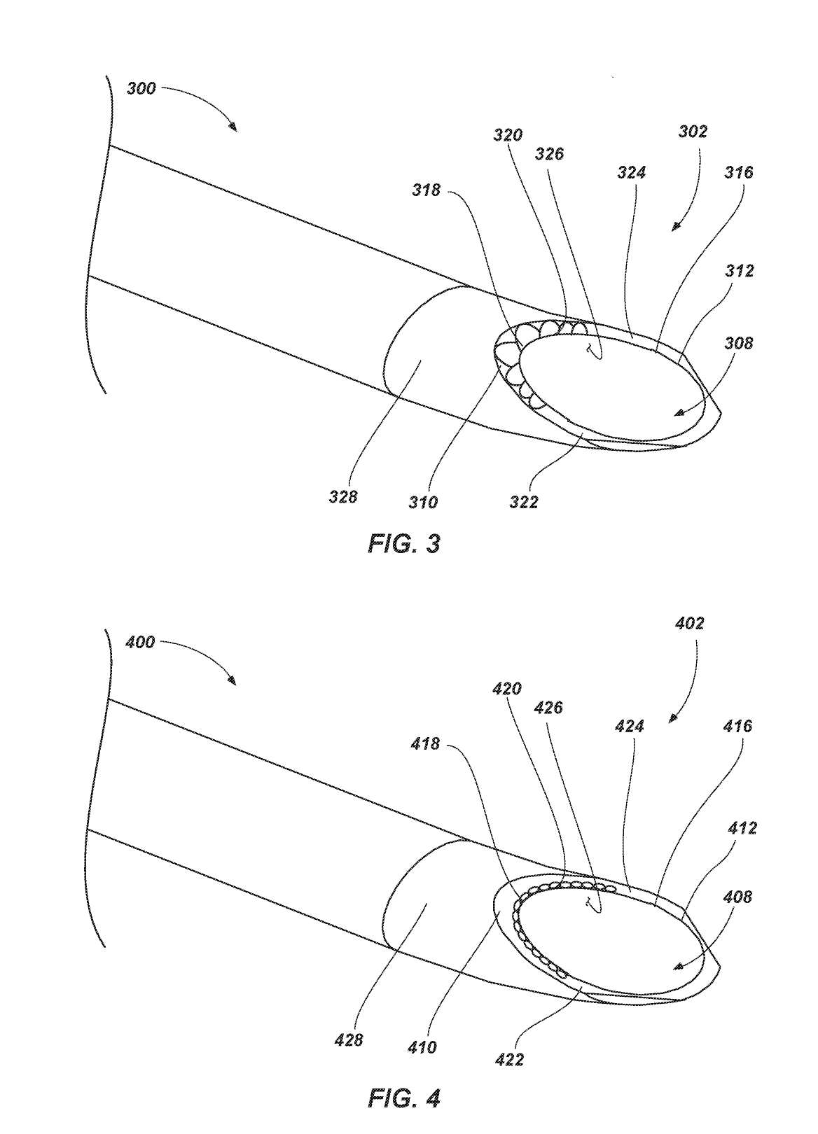

FIG. 3 depicts an enlarged view of a distal portion 302 of an introducer needle 300. The introducer needle 300 may be similar to and include one or more of the same features and functioning, as well as being formed by similar methods, as the introducer needles 100, 200 discussed above with reference to FIGS. 1 and 2. As shown in FIG. 3, the distal portion 302 of the introducer needle 300 may include a bevel 310 formed or shaped to comprise an opening 312 at a distal end of the bore 308 of the introducer needle 300. The distal portion 302 is curved upward to allow fluid being ejected therefrom or any medical devices threaded therethrough to exit in an expected direction.

As depicted, the introducer needle 300 includes one or more features (e.g., smooth surfaces or protrusions 318) formed proximate the opening 312 (e.g., at, bordering, forming a boundary of the opening 316) of the bore 308 at the distal portion 302 of the introducer needle 300. For example, the protrusions 318 may be positioned directly at a proximal surface or end 320 (e.g., the heel or trailing end of the bevel 310) of the portion of the introducer needle 300 defining the opening 312 (e.g., the bevel 310). In some embodiments, the protrusions 318 also at least partially define the opening 312.

As further depicted, the protrusions 318 may comprise a plurality of overlapping portions (e.g., beads) that, together, provide an at least partially rounded or undulating surface on the proximal end 320 of the bevel 310. Such protrusions 318 may be formed through a fabrication process, such as, for example, a welding or other thermal or deposition process. In additional to that discussed above, the multiple rounded surfaces or beads may distribute loads over the rounded surfaces of the protrusions 318, for example, to provide a ball-bearing effect as the medical device comes into contact with and travels along the protrusions 318.

In some embodiments, the protrusions 318 extend into the opening 316, thereby defining at least a portion of the circumference of the opening 316. In some embodiments, the protrusions 318 extend into the opening 316 and extend partially onto an inner surface 326 of the introducer needle 300 defining the bore 308.

As above, the introducer needle 300 employs the addition of material to form the protrusions 318 of the introducer needle 300 proximate the opening 312 to reduce the probability of damaging an associated medical device with the sharp inside edge of the bevel 310. The protrusions 310, which, as depicted, may surround about one-fourth to one-third or more of the opening 312, act to provide a relatively more smooth surface for a medical device to contact and/or move or translate along (e.g., slide) during a procedure (e.g., during deployment and/or retraction from the introducer needle 300) as compared to conventional introducer needles.

As further depicted in FIG. 3, the protrusions 318 may collectively comprise a rounded or undulating surface (e.g., having a substantially wedge-shape) extending at a junction between the bevel 310 (e.g., a surface defining the bevel 310) and the inner surface 326 of the introducer needle 300 defining the bore 308 from a first lateral side 322 of the opening 312, along the proximal end 320 of the opening 312, and to a second lateral side 324 of the opening 312 (e.g., opposing the first lateral side 322). In some embodiments, the protrusions 318 may extend from a junction between the bevel 310 and the inner surface 326 of the introducer needle 300 to (e.g., stopping at or continuing past) another junction between an outer surface 328 of the introducer needle 300 (e.g., an outer circumferential surface of the introducer needle 300) and the bevel 310.

FIG. 4 depicts an enlarged view of a distal portion 402 of an introducer needle 400. The introducer needle 400 may be similar to and include one or more of the same features and functioning, as well as being formed by similar methods, as the introducer needles 100, 200, 300 discussed above with reference to FIGS. 1 through 3. As shown in FIG. 4, the distal portion 402 of the introducer needle 400 may include a bevel 410 formed or shaped to comprise an opening 412 at a distal end of the bore 408 of the introducer needle 400. The distal portion 402 is curved upward to allow fluid being ejected therefrom or any medical devices threaded therethrough to exit in an expected direction.

As depicted, the introducer needle 400 includes one or more features (e.g., smooth surfaces or protrusions 418) formed proximate the opening 412 (e.g., at, bordering, forming a boundary of the opening 416) of the bore 408 at the distal portion 402 of the introducer needle 400. For example, the protrusions 418 may be positioned directly at a proximal surface or end 420 (e.g., the heel or trailing end of the bevel 410) of the portion of the introducer needle 400 defining the opening 412 (e.g., the bevel 410). In some embodiments, the protrusions 418 also at least partially define the opening 412.

As further depicted, the protrusions 418 may comprise a plurality of rounded surface features (e.g., beads) that, together, provide an at least partially rounded surface on the proximal end 420 of the bevel 410. Such protrusions 418 may be formed through a fabrication process, such as, for example, a welding or other thermal or deposition process. In addition to that discussed above, the plurality of rounded surface features or beads may distribute loads over the rounded surfaces of the protrusions 418, for example, to provide a ball-bearing effect as the medical device comes into contact with and travels along the protrusions 418.

In some embodiments, the protrusions 418 extend into the opening 416, thereby defining at least a portion of the circumference of the opening 416. In some embodiments, the protrusions 418 extend into the opening 416 and extend partially onto an inner surface 426 of the introducer needle 400 defining the bore 408.

As above, the introducer needle 400 employs the addition of material to form the protrusions 418 of the introducer needle 400 proximate the opening 412 to reduce the probability of damaging an associated medical device with the sharp inside edge of the bevel 410. The protrusions 418, which, as depicted, may surround about one-third to one-half or more of the opening 412, act to provide a relatively more smooth surface for a medical device to contact and/or move or translate along (e.g., slide) during a procedure (e.g., during deployment and/or retraction from the introducer needle 400) as compared to conventional introducer needles.

As further depicted in FIG. 4, the protrusions 418 may collectively comprise a rounded surface (e.g., having a substantially wedge-shape) extending at a junction between the bevel 410 (e.g., a surface defining the bevel 410) and the inner surface 426 of the introducer needle 400 defining the bore 408 from a first lateral side 422 of the opening 412, along the proximal end 420 of the opening 412, and to a second lateral side 424 of the opening 412 (e.g., opposing the first lateral side 422). In some embodiments, the protrusions 418 may extend from a junction between the bevel 410 and the inner surface 426 of the introducer needle 400 to (e.g., stopping at or continuing past) another junction between an outer surface 428 of the introducer needle 400 (e.g., an outer circumferential surface of the introducer needle 400) and the bevel 410.

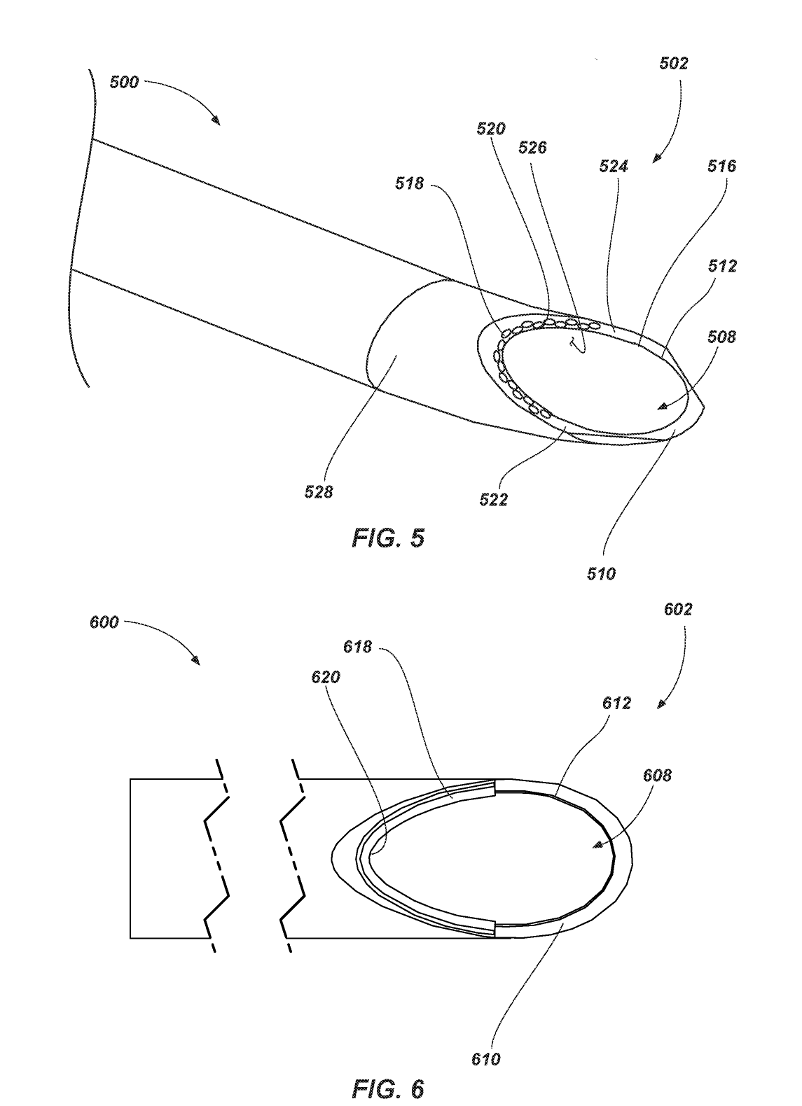

FIG. 5 depicts an enlarged view of a distal portion 502 of an introducer needle 500. The introducer needle 500 may be similar to and include one or more of the same features and functioning, as well as being formed by similar methods, as the introducer needles 100, 200, 300, 400 discussed above with reference to FIGS. 1 through 4. As shown in FIG. 5, the distal portion 502 of the introducer needle 500 may include a bevel 510 formed or shaped to comprise an opening 512 at a distal end of the bore 508 of the introducer needle 500. The distal portion 502 is curved upward to allow fluid being ejected therefrom or any medical devices threaded therethrough to exit in an expected direction.

As depicted, the introducer needle 500 includes one or more features (e.g., smooth surfaces or protrusions 518) formed proximate the opening 512 (e.g., at, bordering, forming a boundary of the opening 516) of the bore 508 at the distal portion 502 of the introducer needle 500. For example, at least some of the protrusions 518 may be positioned directly at a proximal surface or end 520 (e.g., the heel or trailing end of the bevel 510) of the portion of the introducer needle 500 defining the opening 512 (e.g., the bevel 510). In some embodiments, at least some of the protrusions 518 also at least partially define the opening 512.

As further depicted, the protrusions 518 may comprise a plurality of rounded surface features (e.g., beads) that, together, provide an at least partially rounded surface on the proximal end 520 of the bevel 510. The distance of the protrusions 518 from the opening 516 may vary along the protrusions 518. For example, alternating protrusions 518 may be spaced relatively further away from the opening 516 as compared to one or more adjacent protrusions 518. Such protrusions 518 may be formed through a fabrication process, such as, for example, a welding or other thermal or deposition process. In additional to that discussed above, the plurality of rounded surface features or beads may distribute loads over the rounded surfaces of the protrusions 518, for example, to provide a ball-bearing effect as the medical device comes into contact with and travels along the protrusions 518.

In some embodiments, at least some of the protrusions 518 extend into the opening 516, thereby defining at least a portion of the circumference of the opening 516. In some embodiments, at least some of the protrusions 518 extend into the opening 516 and extend partially onto an inner surface 526 of the introducer needle 500 defining the bore 508.

As above, the introducer needle 500 employs the addition of material to form the protrusions 518 of the introducer needle 500 proximate the opening 512 to reduce the probability of damaging an associated medical device with the sharp inside edge of the bevel 510. The protrusions 518, which, as depicted, may surround about one-third to one-half or more of the opening 512, act to provide a relatively more smooth surface for a medical device to contact and/or move or translate along (e.g., slide) during a procedure (e.g., during deployment and/or retraction from the introducer needle 500) as compared to conventional introducer needles.

As further depicted in FIG. 5, the protrusions 518 may collectively comprise a rounded surface (e.g., having a substantially wedge-shape) extending at or proximate a junction between the bevel 510 (e.g., a surface defining the bevel 510) and the inner surface 526 of the introducer needle 500 defining the bore 508 from a first lateral side 522 of the opening 512, along the proximal end 520 of the opening 512, and to a second lateral side 524 of the opening 512 (e.g., opposing the first lateral side 522). In some embodiments, at least some of the protrusions 518 may extend from a junction between the bevel 510 and the inner surface 526 of the introducer needle 500 toward (e.g., stopping at or continuing past) another junction between an outer surface 528 of the introducer needle 500 (e.g., an outer circumferential surface of the introducer needle 500) and the bevel 510.

FIG. 6 depicts an enlarged view of a distal portion 602 of an introducer needle 600. The introducer needle 600 may be similar to and include one or more of the same features and functioning, as well as being formed by similar methods, as the introducer needles 100, 200, 300, 400, 500 discussed above with reference to FIGS. 1 through 5. As shown in FIG. 6, the distal portion 602 of the introducer needle 600 may include a bevel 610 formed or shaped to comprise an opening 612 at a distal end of the bore 608 of the introducer needle 600. The distal portion 602 is curved upward to allow fluid being ejected therefrom or any medical devices threaded therethrough to exit in an expected direction.

As depicted, the introducer needle 600 includes one or more features (e.g., a smooth surface or protrusion 618) formed proximate the opening 612 (e.g., at, bordering, forming a boundary of the opening 616) of the bore 608 at the distal portion 602 of the introducer needle 600. For example, the protrusion 618 may be positioned directly at a proximal surface or end 620 (e.g., the heel or trailing end of the bevel 610) of the portion of the introducer needle 600 defining the opening 612 (e.g., the bevel 610). In some embodiments, the protrusion 618 also at least partially defines the opening 612.

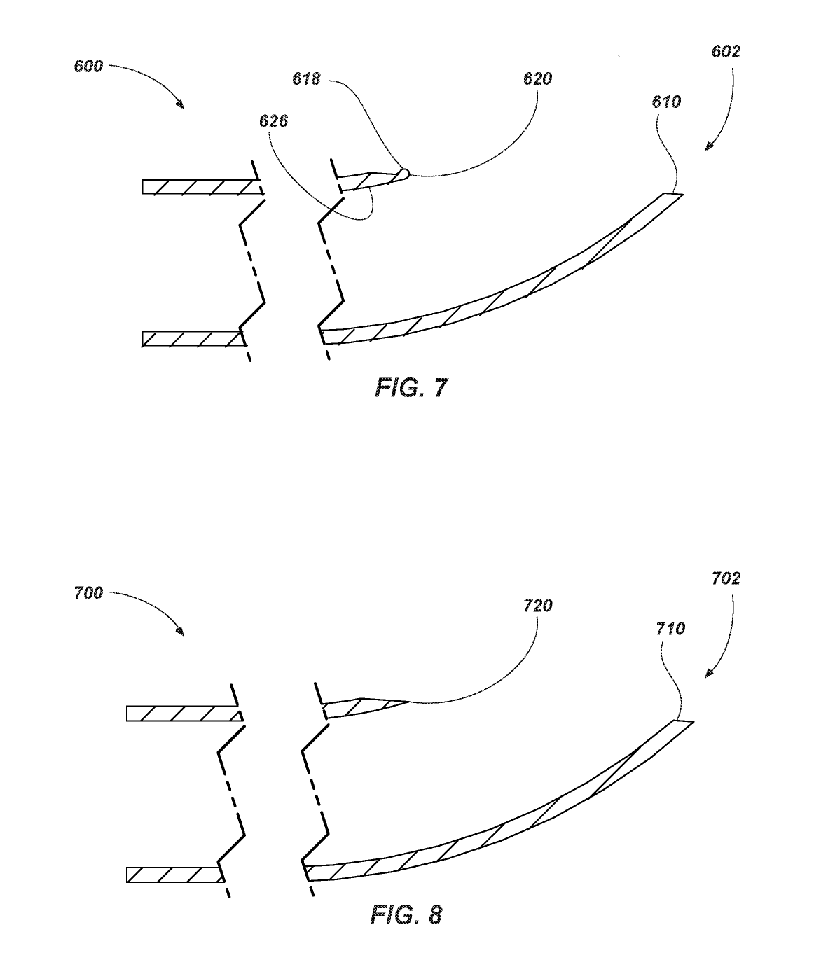

FIG. 7 depicts a cross-sectional view of the distal portion 602 of the introducer needle 600 of FIG. 6. As shown in FIG. 7, the protrusion 618 may be formed as an at least partially rounded surface of the proximal end 620 of the bevel 610. As above, the introducer needle 600 employs the addition of material to form the protrusion 618 on the introducer needle 600 proximate the opening 612 to reduce the probability of damaging an associated medical device with the sharp inside edge of the bevel 610. The protrusions 618, which, as depicted in FIG. 6, may surround about one-third to one-half or more of the opening 612, act to provide a relatively more smooth surface for a medical device to contact and/or move or translate along (e.g., slide) during a procedure (e.g., during deployment and/or retraction from the introducer needle 600) as compared to conventional introducer needles.

As further depicted in FIGS. 6 and 7, the protrusion 618 may define a rounded surface (e.g., having a substantially wedge-shape) extending at or proximate a junction between the bevel 610 (e.g., a surface defining the bevel 610) and the inner surface 626 of the introducer needle 600 defining the bore 608.

FIG. 8 depicts a cross-sectional view of a distal portion 702 of a conventional introducer needle 700. As shown in FIG. 8, the distal portion 702 of the introducer needle 700 includes a sharp cutting surface on the proximal end 720 of the bevel 710, as discussed hereinabove.

FIG. 9 depicts an enlarged cross-sectional enlarged view of a portion of the distal portion 602 of the introducer needle 600 of FIG. 6 superimposed over an enlarged cross-sectional enlarged view of a portion of the distal portion 702 of the introducer needle 700 of FIG. 8 in order to illustrate the difference between an embodiment of an introducer needle of the instant disclosure and a conventional introducer needle. As shown in FIG. 9, the sharp cutting surface on the proximal end 720 of the conventional introducer needle 700 is replaced (e.g., modified to become) with the at least partially rounded surface of the protrusion 618 at the proximal end 620 of the bevel 610.

As discussed above, and a shown in FIG. 10 (illustrating a medical device 800 being deployed from the distal portion 602 of the needle 600), for example, such a surface at the proximal end 620 of the bevel 610 (e.g., the at least partially rounded surface of the protrusion 618) may act to provide a relatively more smooth surface for the medical device 800 to contact and/or move or translate along (e.g., slide) during a procedure (e.g., during deployment and/or retraction from the introducer needle 600) as compared to conventional introducer needles (e.g., introducer needle 700 (FIGS. 8 and 9)).

Referring back to FIG. 2, a distal portion of the introducer needle 200 may include one or more rounded, beveled, or otherwise chamfered surfaces. For example, a distalmost end 230 (e.g., terminal end) of the introducer needle 200 may include chamfered surfaces 232 extending between the bevel 210 and an outer surface 234 (e.g., a rounded outer surface, an outer circumferential surface) of the introducer needle 200.

The chamfered surfaces 232 may comprise, for example, rounded surfaces, smoothed surfaces, linear chamfered or beveled surfaces, and/or surfaces modified to exhibit an outer angle of substantially less than 90 degrees (e.g., 20 to 70 degrees). As depicted, the chamfered surfaces 232 may be positioned on a side of the bevel 210 opposing the protrusion 218 (e.g., on a distal portion of the bevel 210, a leading or forward rounded edge of the introducer needle 200). For example, the chamfered surfaces 232 may be positioned on either side of a tip 236 (e.g., sharp tip) of the introducer needle 200 configured to penetrate tissue as the introducer needle 200 is inserted within a subject. In such an embodiment, the chamfered surfaces 232 may act to reduce the size (e.g., width) of the sharp, cutting tip 236 of the introducer needle 200. The chamfered surfaces 232 may act as a piercing point (e.g., a substantially non-cutting surface) that penetrates tissue of the subject, substantially without cutting and assists in forming a puncture hole.

In some embodiments, the chamfered surfaces 232 are positioned such that only a relatively small cutting point remains at the tip 236 of the introducer needle 200.

In some embodiments, and as depicted, the introducer needle 200 includes both the chamfered surfaces 232 and a protrusion or protrusions (e.g., protrusion(s) 218, 318, 418, 518 as discussed above with reference to FIGS. 2 through 5). In other embodiments, the introducer needle 200 includes one of the chamfered surfaces 232 and a protrusion or protrusions.

In some embodiments, the chamfered surfaces 232 are formed by grinding and/or polishing the introducer needle 200.

The chamfered surfaces 232 may exhibit a substantially linear and/or rounded shape at various angles extending between the bevel 210 and the outer surface 234 of the introducer needle 200. In some embodiments, the configuration and angle of each of the chamfered surfaces 232 is selected to modify the penetration force of the introducer needle 200 to a desired level depending on clinical conditions and/or practitioner preference.

Embodiments of introducer needles including one or more chamfered surfaces may provide an introducer needle having a sharp tip so as to penetrate tissue planes upon entry. However, many conventional introducer needles have a relatively wide cutting edge because of the large opening of the orifice or bore formed in the introducer needle needed to introduce catheters and leads, which relatively wide cutting edge is traumatic to tissues. Such a relatively wide cutting edge may act like a scalpel edge with a wide cutting surface. Embodiments of introducer needles including chamfered surfaces may act to minimize the tissue damage as compared to conventional introducer needles lacking such chamfered surfaces. Embodiments of such needles may tend to spread tissues of a subject, rather than cutting the tissues, when the needle is inserted into the subject.

Embodiments of introducer needles including one or more chamfered surfaces may further provide an introducer needle with a smaller cutting tip that will reduce (or further reduce when used in conjunction with the protrusion(s) discussed above) the chances of shearing, cutting, and/or skiving of a medical device being used in conjunction with the introducer needle (e.g., being deployed through the introducer needle). For example, even in the event that a medical device being deployed through the introducer needle (e.g., a polymer catheter or lead) was to bend in an unintended direction, against the leading tip of the needle (e.g., in a direction extending around and toward the sharp tip of the needle), rather than away from the sharp tip in a direction dictated by the bevel of the introducer needle, the catheter or lead may still glide along the chamfered surfaces as the catheter or lead will tend to move to one of the chamfered surfaces and away from the sharp tip. For example, as shown in the FIG. 10, medical device 800 may come into contact with a leading tip 636 of the needle 600. As the chamfer surfaces provide a relatively smooth interface surface, there is significantly less probability of the catheter or lead being damaged by the chamfered surfaces.

Once being apprised of the instant disclosure, one of ordinary skill in the art will be able to make and use the devices and assemblies disclosed herein.

What is claimed is:

1. An introducer needle, comprising:
a cannula defining a bore within the cannula, the bore configured to pass at least one associated medical device through the bore and into a subject;
a beveled end defining a distal opening of the bore of the cannula; and
at least one protrusion on a proximal surface of the beveled end proximate the distal opening.

2. The introducer needle of claim 1, wherein the introducer needle further comprises at least one chamfered surface extending between a surface defining the beveled end and an outer circumferential surface of the cannula and positioned proximate a sharp distal tip of the beveled end of the introducer needle.

3. The introducer needle of claim 2, wherein the at least one chamfered surface comprises two chamfer surfaces positioned on either side of the sharp distal tip of the beveled end of the introducer needle.

4. The introducer needle of claim 1, wherein the at least one protrusion comprises an at least partially rounded surface.

5. The introducer needle of claim 1, wherein the at least one protrusion comprises a rounded protrusion having a substantially wedge-shape extending from a first lateral side of the distal opening, around a proximal portion of the distal opening, and to a second lateral side of the distal opening opposing the first lateral side.

6. The introducer needle of claim 1, wherein the at least one protrusion comprises beads collectively having a substantially wedge-shape extending from a first lateral side of the distal opening, around a proximal portion of the distal opening, and to a second lateral side of the distal opening opposing the first lateral side.

7. The introducer needle of claim 6, wherein each bead of the beads overlaps with at least one adjacent bead of the beads.

8. The introducer needle of claim 6, wherein a spacing of each bead of the beads from the distal opening is offset relative to a spacing of at least one adjacent bead of the beads from the distal opening.

9. The introducer needle of claim 1, wherein the beveled end is positioned in a plane that is positioned at an oblique angle relative to a longitudinal axis of the cannula.

10. The introducer needle of claim 1, wherein the at least one protrusion extends onto an interface between a surface defining the beveled end and an inner surface of the cannula.

11. A needle comprising a cannula defining a bore within the cannula, a beveled end defining a distal opening of the bore of the cannula and having a sharp tip, and at least one chamfered surface extending between a surface defining the beveled end and an outer circumferential surface of the cannula and positioned proximate the sharp tip.

12. The needle of claim 11, wherein the at least one chamfered surface comprises two chamfer surfaces positioned on either side of the sharp tip of the beveled end of the introducer needle.

13. The needle of claim 11, further comprising at least one protrusion on a proximal surface of the beveled end proximate the distal opening.

14. A needle, comprising:
a cannula defining a bore within the cannula, the bore configured to pass at least one associated medical device through the bore and into a subject;
a distal portion defining a distal opening of the bore of the cannula; and
at least one enlarged rounded surface on the distal portion proximate the distal opening.

15. The needle of claim 14, wherein the distal opening is positioned in a plane that is positioned at an oblique angle relative to a longitudinal axis of the cannula.

16. The needle of claim 14, wherein the at least one enlarged rounded surface comprises at least one rounded protrusion positioned on a proximal portion of the distal portion of the needle defining a rearward portion of the distal opening.

17. A medical device assembly comprising:
a medical device configured to have at least a portion thereof positioned within subcutaneous tissue of a subject; and
the needle of claim 1 for inserting at least a portion of the medical device within the subject.

18. A method of forming a needle, the method comprising.
providing a cannula having a bore within the cannula;
configuring the bore to pass at least one associated medical device through the bore and into a subject;
defining a distal opening of the bore of the cannula at a distal end of the needle; and
forming at least one protrusion on a distal portion of the cannula bordering the distal opening of the cannula.

19. The method according to claim 18, wherein forming the at least one protrusion comprises utilizing a welding process to define the at least one protrusion.

20. The method according to claim 18, wherein forming the at least one protrusion comprises defining the at least one protrusion at a proximal portion of the distal opening.
